(19) United States
(12) Patent Application Publication
(10) Pub. No.: US 2017/0105392 A1
(43) Pub. Date: Apr. 20, 2017

(54) METHOD FOR SELECTIVE BREEDING OF FAST-GROWING STRAIN OF LINED SEAHORSE

(71) Applicant: SOUTH CHINA SEA INSTITUTE OF OCEANOLOGY, CHINESE ACADEMY OF SCIENCES, Guangzhou (CN)

(72) Inventors: Qiang LIN, Guangzhou (CN); Wei LUO, Guangzhou (CN); Geng QIN, Guangzhou (CN); Huixian ZHANG, Guangzhou (CN); Yanhong ZHANG, Guangzhou (CN)

(73) Assignee: SOUTH CHINA SEA INSTITUTE OF OCEANOLOGY, CHINESE A CADEMY OF SCIENCES, Guangzhou (CN)

(21) Appl. No.: 15/128,110

(22) PCT Filed: Jun. 15, 2015
(86) PCT No.: PCT/CN2015/081437

§ 371 Date: Sep. 22, 2016

(30) Foreign Application Priority Data
Mar. 9, 2015 (CN) 201510103327.6

(51) Int. Cl. A01K 61/10; A01K 61/95; A01K 67/02

(52) U.S. Cl. CPC A01K 61/10; A01K 67/02; A01K 61/95

(57) ABSTRACT

An method for selective breeding of a fast-growing strain of lined seahorse, including: (1) selecting high-quality lined seahorse parents as a base population for selective breeding; (2) performing prenatal intensified breeding; (3) performing pairing and mating, and rearing the juveniles thereof; (4) performing selective breeding in three junctures with different selection proportions; (5) pairing the saved adult lined seahorses, wherein those with low vitality, having injuries on the body surface, or having a dysplastic brood pouch or gonad are culled, and a selection proportion of 10% for every generation is maintained thereafter; (6) repeating steps (2) through (5) for at least four times to obtain a high-quality strain of lined seahorse with improved weight and body length and stable growth. The method can be used for selective breeding of a new strain with high quality and high yield.

### FIELD OF THE INVENTION

The invention relates to the field of breeding of marine medicinal fish, specifically to a method for selective breeding of fast-growing strain of lined seahorse.

### BACKGROUND OF THE INVENTION

Lined seahorse (Hippocampus erectus), which falls under the genus Hippocampus in the family Synagnathidae, mainly lives in the western Atlantic Ocean. It has relative larger body size, better disease resistance and broader temperature adaptation range, and grows faster compared with other seahorse species. Since its introduction into China in 2009, the lined seahorse has become one of the main aquaculture species. However, degeneration of germplasm becomes a severe problem, in terms of decreased body size, precocious puberty, and reduction of growth rate, reproduction rate and disease resistance after several generations of hatchery (Lin, et al., 2012).

High-quality strain is important to aquaculture and can bring great economic and social benefits to society. Compared with other aquatic animals, very few researches on seahorse have been reported, which mainly focus on the physiological and ecological research of mature seahorse, let alone reports on modern methods for breeding lined seahorse. Genetic improvement of seahorse is very difficult due to its reproductive characteristics, such as male pregnancy, lengthy parental care, small brood size and low birth rate. The quality of the available fries varies, which hampers the promotion of lined seahorse. In view of the above problems, it is necessary to develop a method for selectively breeding lined seahorse to obtain high quality strains and promote the healthy development of the seahorse aquaculture.

### SUMMARY OF THE INVENTION

An objective of the present invention is to provide a practicable and efficient method for selective breeding fast-growing strain of lined seahorse, based on the reproductive characteristics thereof.

The method of the present invention for selective breeding of fast-growing strain of lined seahorse, includes the following steps:

(1) selecting high-quality lined seahorse parents as base population for selective breeding;

(2) performing prenatal intensified breeding on the lined seahorse parents from step (1) at the age of 3-5 months;

(3) pairing and mating the lined seahorse parents from step (2), and carefully rearing the juveniles thereof;

(4) performing selective breeding on the male and female juveniles from step (3) in the following three junctures with different selection proportions: at the age of 2-3 months which is a rapidly growing stage, selecting juveniles with highest growth rate and high vitality; when the selected juveniles grow to a marketable stage at the age of 4-5 months, selecting marketable lined seahorses with highest growth rate and high vitality; when the selected marketable lined seahorses grow to a sexual maturity stage at the age of 6 months, selecting adult lined seahorses with highest growth rate and high vitality;

(5) pairing the adult lined seahorses from step (4), wherein those with low vitality, having injuries on the body surface, or having a dysplastic brood pouch or gonad are culled to maintain a selection proportion of 10% for every generation; and

(6) repeating steps (2) through (5) for at least four times to obtain a high-quality strain of lined seahorses with greatly improved weight and body length and stable growth.

The high-quality lined seahorse parents in step (1) are preferably collected from different geographical populations in USA, at least 100 pairs.

The prenatal intensified breeding in step (2) preferably comprises the following steps: rearing the parents in a parental pool with a stable water temperature between 24° C. to 30° C.; feeding the parents, at 8 am, 1 pm and 6 pm respectively every day, with frozen shrimps as a major feed and fairy shrimps as a supplement, wherein an amount of the frozen shrimps is 5%-10% of the total weight of the lined seahorse, and an amount of the fairy shrimps is 3%˜8% of the total weight of the lined seahorse.

The pairing and mating in step (3) preferably comprises the following steps: transferring the lined seahorses into an ecological simulation white barrel for artificial pairing and mating under ecological simulation condition when male and female lined seahorses show estrus by chasing each other. The ecological simulation white barrel is an apparatus for simulating the mating ecological environment of lined seahorse, with a proper quantity of weeds and branches in it, proper illumination of 3000 to 6000 Lx, and sustainment of slow flow for stimulation.

The rearing the juveniles in step (3) preferably comprises the following steps: collecting newly hatched juveniles after they are released by male lined seahorses, rearing them in a small cement pool with fairy shrimps as a major feed and an amount of the fairy shrimps is 3%˜8% of the total weight of the juveniles; at the age of 2˜3 months, transferring the juveniles into a breeding pond, and feeding them with fairy shrimps and frozen shrimps, wherein an amount of the frozen shrimps is 5%˜10% of the total weight of the juveniles, and an amount of the fairy shrimps is 3%˜8% of the total weight of the juveniles; feeding the adult lined seahorses with frozen shrimps as a major feed and fairy shrimps as a supplement, wherein an amount of the frozen shrimps is 5%˜10% of the total weight of the lined seahorse, and an amount of the fairy shrimps is 3%˜8% of the total weight of the lined seahorse.

The selection proportions in step (4) preferably are 60% at the age of 2-3 months, 50% at the age of 4-5 months, and 40% at the age of 2-3 months.

Preferably, in step (5), the lined seahorses with low vitality, having injuries on the body surface, or having a dysplastic brood pouch or gonad are culled based on a selection proportion of 80%˜90%.

After selective breeding for 5 generations with the method of the present invention, a selective breeding strain and a nonselective breeding strain were artificially paired at the same time.

All progenies of the two strains were reared under a same condition, and the body weights and lengths thereof were measured at the age of 2 months, 4 months and 6 months, respectively. At the age of 6 months, the weight of the progenies of the selective breeding strain was about 14% higher than those of the nonselective breeding strain.

The selective breeding method of the present invention can be used for selective breeding of a new strain with high quality and high yield, and in the germplasm level overcome the low growth rate and irregular size of the lined seahorse.

### BRIEF DESCRIPTION OF THE DRAWINGS

FIG. 1 shows comparison of the body weight at the age of 6 months between the selective breeding strain and the nonselective breeding strain.

FIG. 2 shows comparison of the body length at the age of 6 months between the selective breeding strain and the nonselective breeding strain.

### DETAILED DESCRIPTION OF THE EMBODIMENTS

Embodiment below is provided for further illustration but without limiting the present invention.

### Embodiment 1

(1) Introduction of High-Quality Lined Seahorses from USA, and Intensified Breeding Thereof as Parents.

In 2009, our team introduced crossing progenies of various geological populations of lined seahorses from different places such as Florida and North Carolina, totally 100 pairs at the age of about 4 months with their body length of 9˜14 cm and body weight of 3˜6 g. The parents were breeding in a parental pool at a water temperature stabilized at 24˜30 C., and fed with frozen shrimps as a major feed and fairy shrimps as a supplement three times every day at 8 am, 1 pm and 6 pm respectively, wherein a daily amount of the frozen shrimps is 5%˜10% of the total weight of the lined seahorse, and an amount of the fairy shrimps is 3%˜8% of the total weight of the lined seahorse. The lined seahorses were the base population for selective breeding of fast-growing strain.

(2) Artificial Pairing and Mating Under Ecological Simulation Condition, Rearing of Juveniles, and Breeding of Adults.

The first artificial mating was performed on Mar. 8 to 15, 2010. Aquaculture water in the ecological simulation white barrels was maintained at a temperature of 24˜30° C., a salinity of 20˜32‰ and a pH of 7.8˜8.4, with illumination of 3000 to 6000 Lx, and sustainment of slow flow for stimulation. A few gulfweeds or other weeds were placed in the white barrels for the lined seahorses to latch on with their prehensile tails so as to facilitate perching. When the male and female lined seahorses in the parental pool showed estrus by chasing each other, they were transferred to the white barrels for artificial pairing and mating under ecological simulation condition, with 5 pairs of female and male lined seahorses in each barrel. The females released eggs into the brood pouches of the males, and after 15˜20 days of pregnancy, the males released juveniles. On March 26 to April 5, the newly hatched juveniles were collected and transferred into a small cement pool (length: 3 m, width: 3 m, height: 1 m) for rearing them. The juveniles were fed mainly with fairy shrimps, and an amount of the fairy shrimps was 3%˜8% of the total weight of the juveniles. When they grew to the age of 2˜3 months, they were transferred into breeding ponds (length: 5 m, width: 5 m, height: 1 m) and fed with frozen shrimps and fairy shrimps, wherein an amount of the frozen shrimps was 5%˜10% of the total weight of the juveniles, and an amount of the fairy shrimps was 3%˜8% of the total weight of the juveniles. When they grew to the age of 4 months, they were fed with frozen shrimps as a major feed and fairy shrimps as a supplement, wherein an amount of the frozen shrimps was 5%˜10% of the total weight of the lined seahorse, and an amount of the fairy shrimps is 3%˜8% of the total weight of the lined seahorse.

(3) Selections in F₁ Generation of the Selective Breeding Strain at Different Growth Stages

Selections were performed at rapidly growing stage (at the age of 2˜3 months), marketable stage (at the age of 4˜5 months) and sexual maturity stage (at the age of 6 months) respectively. Before each selection, we measured random samples of the lined seahorses, and then determined the cut-off value (body weight) for the selections based on the selection proportions.

On June 14, the first selection was performed. About 12 thousand juveniles randomly reared in ecological simulation white barrels were subjected to the selection, wherein juveniles with high growth rate and high vitality were saved based on a selection proportion of 60% with a cut-off value of 0.4 g (body weight). The saved juveniles, with a number of about 7000, were then transferred and reared in a large ecological simulation cement pool.

On August 20, the second selection was performed. 2 months after the first selection, the saved juveniles grew to a marketable size. About 6000 juveniles were subjected to the selection wherein juveniles with high growth rate and high vitality were saved based on a selection proportion of 50% with a cut-off value of 6.0 g (body weight). The saved juveniles, with a number of about 3000, were then transferred and reared in another large ecological simulation cement pool.

On October 10, the third selection was performed. 2 months after the second selection, the saved juveniles reached sexual maturity. About 3000 juveniles were subjected to the selection, wherein juveniles with high growth rate and high vitality were saved based on a selection proportion of 40% with a cut-off value of 11.0 g (body weight). The saved juveniles, with a number of about 1100, were then transferred and reared in a third large ecological simulation cement pool.

From October 18 to 25, artificial pairing and mating were performed. We randomly divided the saved lined seahorses after the three selections for pairing, 5 females and 5 males in each group. They were bred in ecological simulation white barrels with the same method in step (2), and it should be noticed that, those with low vitality, having injuries on the body surface, or having a dysplastic brood pouch or gonad shall be culled under a culling rate of 10˜20%, to maintain a selection proportion of 10% for every generation.

(4) Repeating Steps (2) Through (3) for 4 Times

With the method of the steps (2) and (3), artificial reproductions were performed from May 18 to 26, 2011 to obtain a F₃ generation of the selective breeding strain, from Jan. 7 to 15, 2012 to obtain a F₄ generation thereof, and from Aug. 20 to 28, 2012 to obtain a F₅ generation thereof.

(5) Comparisons

From September, 2016 on, lined seahorses of the nonselective strain, the F₁ generation, the F₂ generation, the F₃ generation, the F₄ generation and the F₅ generation born within one week were collected, and were reared separately in cement pools with the same environment, wherein the stocking density, the feeding and the aquaculture management were all identical. Three parallel experiments were carried out for each generation.

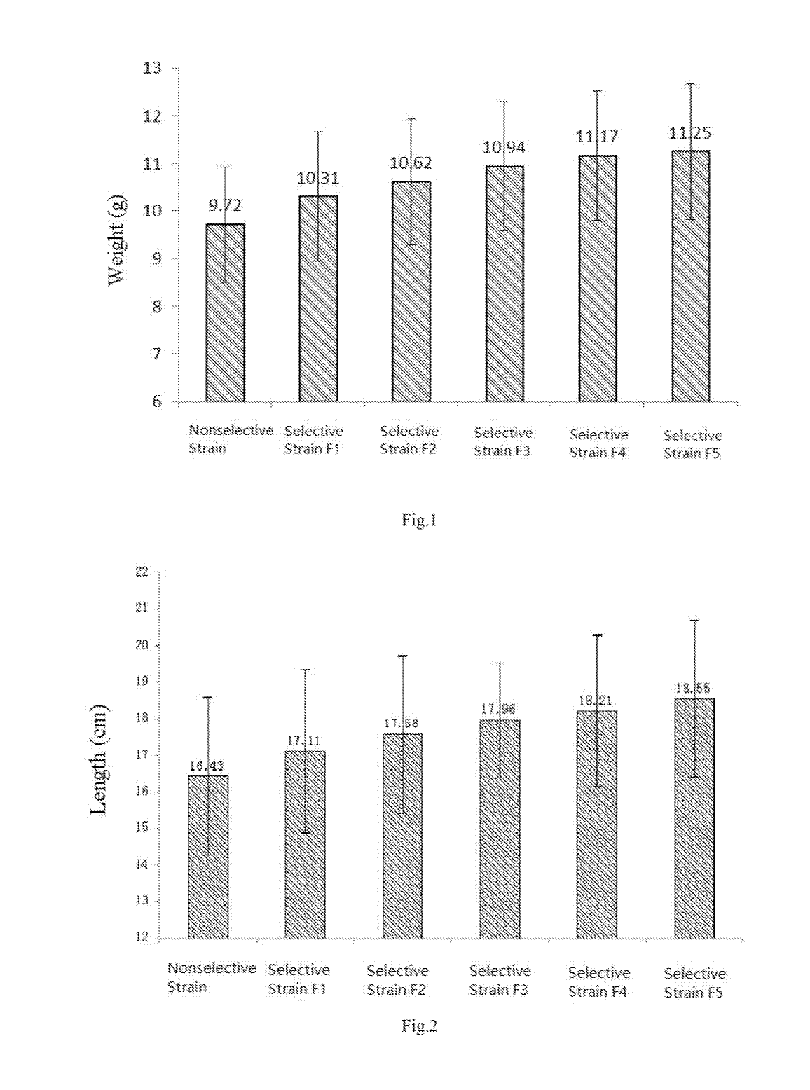

Growth related traits, mainly referred as body weight and body length of the lined seahorse, were measured respectively, on November 2012, January 2013, and April 2013. Table 1 and Table 2 show the weight and length of the selective breeding strains (F₁ to F₅) and the nonselective strain. FIG. 1 and FIG. 2 show the comparison thereof.

TABLE 1
Weight of the lined seahorse at the ages of 2 months, 4 months and 6 months

| | Selective breeding strain | | | | | |
|---|---|---|---|---|---|---|
| | (weight: mean value ± standard deviation, g) | | | | | |
| | Nonselective | | | | | |
| Age | strain | F₁ | F₂ | F₃ | F₄ | F₅ |
| 2-month | 0.34 ± 0.06 | 0.37 ± 0.05 | 0.38 ± 0.07 | 0.40 ± 0.08 | 0.41 ± 0.06 | 0.42 ± 0.07 |
| 4-month | 5.37 ± 0.64 | 5.59 ± 0.82 | 5.87 ± 0.74 | 6.02 ± 0.57 | 6.15 ± 0.92 | 6.20 ± 0.89 |
| 6-month | 9.72 ± 1.21 | 10.31 ± 1.36 | 10.62 ± 1.32 | 10.94 ± 1.35 | 11.17 ± 1.36 | 11.25 ± 1.43 |
| Rise compared with | — | 5.72% | 8.47% | 11.15% | 12.98% | 13.60% |
| nonselective strain at | | | | | | |
| the age of 6 months | | | | | | |

TABLE 2
Length of the lined seahorse at the ages of 2 months, 4 months and 6 months

| | Selective breeding strain | | | | | |
|---|---|---|---|---|---|---|
| | (standard length: mean value ± standard deviation, cm) | | | | | |
| | Nonselective | | | | | |
| Age | strain | F₁ | F₂ | F₃ | F₄ | F₅ |
| 2-month | 5.78 ± 0.39 | 6.01 ± 0.58 | 6.13 ± 0.95 | 6.33 ± 0.75 | 6.43 ± 0.88 | 6.50 ± 0.72 |
| 4-month | 12.35 ± 2.04 | 12.86 ± 1.76 | 13.24 ± 2.03 | 13.42 ± 1.79 | 13.74 ± 2.16 | 13.94 ± 2.17 |
| 6-month | 16.43 ± 2.16 | 17.11 ± 2.23 | 17.58 ± 2.15 | 17.96 ± 1.57 | 18.21 ± 2.07 | 18.55 ± 2.14 |
| Rise compared with | — | 3.97% | 6.54% | 8.52% | 9.77% | 11.43% |
| nonselective strain at | | | | | | |
| the age of 6 months | | | | | | |

1. A method for selective breeding of a fast-growing strain of lined seahorses, comprising:
step (1), selecting high-quality lined seahorse parents as a base population for selective breeding;
step (2), performing prenatal intensified breeding on the lined seahorse parents from the step (1) at an age of 3-5 months;
step (3), pairing and mating the lined seahorse parents from the step (2), and rearing reproduced juveniles thereof;
step (4), performing selective breeding on male and female juveniles from the step (3) in the following three junctures with different selection proportions: at an age of 2-3 months which is a fast-growing stage, selecting juveniles with a highest growth rate and high vitality; when selected juveniles grow to a marketable stage at an age of 4-5 months, selecting marketable juveniles with a highest growth rate and high vitality; when selected marketable juveniles grow to a sexual maturity stage at an age of 6 months, selecting adult lined seahorses with a highest growth rate and high vitality;
step (5), pairing the adult lined seahorses from step (4), wherein those with low vitality, having injuries on a body surface, or having a dysplastic brood pouch or gonad are culled to maintain a selection proportion of 10% for every generation; and
step (6), repeating the steps (2) through (5) for at least four times to obtain a high-quality strain of lined seahorses with an improved weight and body length and a stable growth.

2. The method for selective breeding of a fast-growing strain of lined seahorses according to claim 1, wherein at least 100 pairs of the high-quality lined seahorse parents in the step (1) are collected from different geographical populations in USA.

3. The method for selective breeding of a fast-growing strain of lined seahorses according to claim 1, wherein the prenatal intensified breeding in the step (2) further comprises: rearing the parents in a parental pool with a stable water temperature between 24° C. to 30° C.; feeding the parents, at 8 am, 1 pm and 6 pm respectively every day, with frozen shrimps as a major feed and fairy shrimps as a supplement, wherein an amount of the frozen shrimps is 5%-10% of a total weight of the lined seahorses, and an amount of the fairy shrimps is 3%˜8% of the total weight of the lined seahorses.

4. The method for selective breeding of a fast-growing strain of lined seahorses according to claim 1, wherein the pairing and mating in the step (3) further comprises: transferring the lined seahorses into an ecological simulation white barrel for artificial pairing and mating under an ecological simulation condition when male and female lined seahorses show estrus by chasing each other; the ecological simulation white barrel is an apparatus for simulating a mating ecological environment of the lined seahorses, with a proper quantity of weeds and branches, proper illumination of 3000 to 6000 Lx, and sustainment of a slow flow for stimulation.

5. The method for selective breeding of a fast-growing strain of lined seahorses according to claim 1, wherein the rearing the juveniles in the step (3) further comprises: collecting newly hatched juveniles after they are released by male lined seahorses, rearing them in a small cement pool with fairy shrimps as a major feed and an amount of the fairy shrimps is 3%˜8% of a total weight of the juveniles; at the age of 2˜3 months, transferring the juveniles into a breeding pond, and feeding them with fairy shrimps and frozen shrimps, wherein an amount of the frozen shrimps is 5%˜10% of the total weight of the juveniles, and an amount of the fairy shrimps is 3%˜8% of the total weight of the juveniles; feeding the adult lined seahorses with frozen shrimps as a major feed and fairy shrimps as a supplement, wherein an amount of the frozen shrimps is 5%˜10% of the total weight of the lined seahorses, and an amount of the fairy shrimps is 3%˜8% of the total weight of the lined seahorses.

6. The method for selective breeding of a fast-growing strain of lined seahorses according to claim 1, wherein the selection proportions in the step (4) are respectively 60% at the age of 2-3 months, 50% at the age of 4-5 months, and 40% at the age of 6 months.

7. The method for selective breeding of a fast-growing strain of lined seahorses according to claim 1, wherein the pairing the adult lined seahorses in the step (5) further comprises: culling the lined seahorses with low vitality, having injuries on a body surface, or having a dysplastic brood pouch or gonad, based on a selection proportion of 80˜90%.

8. The method for selective breeding of a fast-growing strain of lined seahorses according to claim 4, wherein the rearing the juveniles in the step (3) further comprises: collecting newly hatched juveniles after they are released by male lined seahorses, rearing them in a small cement pool with fairy shrimps as a major feed and an amount of the fairy shrimps is 3%˜8% of a total weight of the juveniles; at the age of 2˜3 months, transferring the juveniles into a breeding pond, and feeding them with fairy shrimps and frozen shrimps, wherein an amount of the frozen shrimps is 5%˜10% of the total weight of the juveniles, and an amount of the fairy shrimps is 30˜8% of the total weight of the juveniles; feeding the adult lined seahorses with frozen shrimps as a major feed and fairy shrimps as a supplement, wherein an amount of the frozen shrimps is 5%˜10% of the total weight of the lined seahorses, and an amount of the fairy shrimps is 3%˜8% of the total weight of the lined seahorses.
